(19) United States
(12) Patent Application Publication
(10) Pub. No.: US 2023/0313256 A1
(43) Pub. Date: Oct. 5, 2023

(54) MICROBIAL CULTURE MEDIUM

(71) Applicant: LONZA LTD, Visp (CH)

(72) Inventors: Nuria ADELANTADO, Visp (CH); Hauke HOLM, Visp (CH); Christoph KIZIAK, Visp (CH)

(21) Appl. No.: 18/003,991

(22) PCT Filed: Jul. 1, 2021
(86) PCT No.: PCT/EP2021/068207

§ 371 Date: Dec. 30, 2022

(30) Foreign Application Priority Data
Jul. 1, 2020 (EP) 20183422.3

(51) Int. Cl. C12P 21/00; C12N 1/20

(52) U.S. Cl. CPC C12P 21/00; C12N 1/20

(57) ABSTRACT

A method of reducing precipitation in a bacterial fermentation process for producing a fermentation product, which fermentation process comprises the steps:
a) cultivating the host cells for bacterial growth in a batch phase using a batch medium; and
b) cultivating the host cells for producing the fermentation product in a fed-batch phase using a feed medium;
which feed and batch media are each unprecipitated media solutions, wherein:
i) the batch medium comprises an amount of calcium salts, magnesium salts, phosphate salts and at least one chelating agent, which is a citrate salt and/or citric acid at an amount of at least 5 mM, and/or EDTA at an amount of at least 1 mM; and
ii) the feed medium comprises an amount of calcium salts which is at least 1 mM, and which is at least 5-fold higher as compared to the batch medium (M/M); and
iii) the feed medium comprises an amount of magnesium salts which is at least 3 mM, and which is at least 2-fold higher as compared to the batch medium (M/M); and
iv) the feed medium comprises an amount of phosphate salts which is less than 90% of the amount comprised in the batch medium (M/M).

### TECHNICAL FIELD

The invention refers to a method of reducing precipitation in a bacterial fermentation process and a media system for use in a bacterial fermentation process comprising a batch medium and a feed medium

### BACKGROUND

One of the most efficient methods of recombinant protein production in bacterial cells, such as E. coli is fed-batch. During a batch phase, the cells can grow up to very high cell densities. This batch phase is followed by a fed-batch phase, wherein formation of a fermentation product is switched on and the cells are fed to produce the product.

The culture of E. coli cells to high cell densities is an essential prerequisite for high yields of a fermentation product. For this purpose, the cells are growing in an unlimited way (µ=µ ₘₐₓ) in the batch phase to achieve high cell density, and during a subsequent fed-batch phase, a carbon source (e.g., glucose or glycerol) is customarily metered in under substrate-limited conditions. High-cell density fermentation of bacterial cells is an attractive biotechnological means of achieving high space time yields for the production of a fermentation product, such as heterologous proteins.

Fed-batch high density fermentations of E. coli suffer from the disadvantage of moderate to heavy precipitation in the cell culture. The occurrence of precipitation can start already when preparing the cell culture media, e.g., when a medium is prepared at low pH, after heat sterilization or upon setting the pH to the desired value around pH 7.0. So far, the assumption is that even though medium compounds precipitate, they are again dissolved when a compound becomes limiting.

Korz et al. (J. Biotechnol. 1995, 39:59-65) describes a fed-batch technique for high cell density cultivation of E. coli using glucose or glycerol as carbon source. A feed medium is used at a pre-determined feeding rate to maintain carbon-limited growth during the fed-batch process at growth rates that do not cause the formation of acetic acid which is a toxic by-product resulting from incomplete substrate oxidation.

Wilms et al. (Biotechnology and Bioengineering 2001, 73(2):95-103) describe high-cell density fermentation for the production of heterologous proteins in E. coli.

Calleja et al. (Biotechnology and Bioengineering 2016, 113(4):772-782) describe the simulation and prediction of protein production in fed-batch E. coli cultures. Different defined minimal media using glucose as a sole carbon source were used for various strains. A feeding medium composition was used that comprises a phosphate solution in order to avoid co-precipitation with magnesium salts.

Hardiman et al. (J. Biotechnol. 2007, 132:359-374) describe fed-batch cultivation of an E. coli strain and the use of a batch and a fed-batch medium. Phosphoric acid was used to resolve any precipitate.

EP1584680A1 discloses defined cell culture media for use in a fed-batch fermentation process for the production of plasmid DNA. The feeding medium comprises at least the same or higher amounts of phosphate salts compared to the batch medium.

WO95/29986A1 discloses a method for controlling metallophosphate precipitation in high cell density bacterial fermentations using phosphate glass as a source of phosphorus.

WO2018011242A1 discloses a fermentation medium comprising a certain chelating agent.

Shiloach et al. (Biotechnology Advances 2005, 23:345-357) review method development of growing E. coli to high cell density.

Riesenberg et al. (J Biotechnol. 1991, 20:17-28) describe a fed-batch process of high cell density cultivation of E. coli at controlled specific growth rate feeding glucose supplemented with magnesium sulfate.

Precipitates in a bacterial cell culture might have an impact on the robustness of bacterial fermentation processes. Compounds of the precipitates are not available for the cells. Variations in amounts and composition of the precipitates due to different handling procedures or upscaling could lead to variations of nutrient availability for the cells. In addition, it can generate issues for analytics (e.g. optical density (OD) measurements that are disturbed due to the particles), downstream processing (DSP, e.g. clogging of filters), or when a filtration step of the medium is required before its use (e.g. filling vessel for small volume, high throughput fermentation systems after setting the pH). It is thus desirable to reduce precipitation in bacterial cell cultures.

### SUMMARY OF THE INVENTION

It is the object of the invention to provide a fed-batch bacterial cell culture process avoiding precipitation in the cell culture medium, and a respective non-precipitating media system. It is a further object to provide batch and feeding media for high cell density fermentations with bacterial cells, such as E. coli, in which precipitation of medium compounds is prevented in the relevant growing pH range (pH ± 6.7 - 7.3).

The object is solved by the subject matter as claimed and as further described herein.

The invention provides for a method of reducing precipitation in a bacterial fermentation process for producing a fermentation product, which fermentation process comprises the steps:
a) cultivating the host cells for bacterial growth in a batch phase using a batch medium; and
b) cultivating the host cells for producing the fermentation product in a fed-batch phase using a feed medium;
which feed and batch media are each unprecipitated media solutions, wherein:
i) the batch medium comprises an amount of calcium salts, magnesium salts, phosphate salts and at least one chelating agent, which is a citrate salt and/or citric acid at an amount of at least 5 mM, and/or EDTA at an amount of at least 1 mM; and
ii) the feed medium comprises an amount of calcium salts which is at least 1 mM, and which is at least 5-fold higher as compared to the batch medium (M/M), and
iii) the feed medium comprises an amount of magnesium salts which is at least 3 mM, and which is at least 2-fold, or at least 3-fold, or at least 5-fold, or at least 10-fold, or at least 15-fold higher as compared to the batch medium (M/M); and
iv) the feed medium comprises an amount of phosphate salts which is less than 90%, or less than 80%, or less than 70%, or less than 60%, or less than 50%, or less than 40%, or less than 30%, or less than 20%, or less than 10%, or less than any one of 9, 8, 7, 6, 5, 4, 3, 2, or 1%, of the amount comprised in the batch medium (M/M).

According to a specific aspect,
a) the amount of calcium salts is up to 1 mM in the batch medium; and up to 5, or up to 4 mM in the feed medium; and/or
b) the amount of magnesium salts is 1-3 mM in the batch medium; and 3-100 mM, or 30-60 mM in the feed medium; and/or
c) the amount of phosphate salts is 30-120 mM in the batch medium; and in the feed medium less than 90% of the amount comprised in the batch medium (M/M), preferably less than or up to 10 mM, preferably up to 9, up to 8, up to 7, up to 6, up to 5, or up to 4, or up to 3 mM in the feed medium, preferably there are no phosphate salts in the feed medium.

The chelating agent may comprise or consist of either one of a citrate salt, such as sodium citrate, citric acid, or EDTA, or can be a mixture of sodium citrate and citric acid, such as to obtain a total amount of citrate ions, or can be a mixture of EDTA and any one or both of a citrate salt and citric acid.

Specifically, the preferred amount of a chelating agent comprises or consists of a citrate salt, such as sodium citrate, and/or citric acid, at an amount of at least any one of 5, 6, 7, 8, 9, or 10 mM, up to any one of 100, 90, 80, 70, 60, 50, 40, 30, 20, or 10 mM of citrate ions (in total), preferably within a molar range of 6 to 70 mM citrate ions (in total).

Specifically, the preferred amount of a chelating agent comprises or consists of EDTA, such as sodium EDTA, at an amount of at least any one of 1, 2, 3, 4, 5, 6, 7, 8, 9, or 10 mM, up to any one of 30, 29, 28, 27, 26, 25, 24, 23, 22, 21, or 20 mM, preferably within a molar range of 1.5 to 22 mM.

According to a specific aspect, the batch medium further comprises all nutrients, supplements, and excipients as necessary to grow the bacterial cells to high density.

Specifically, the batch medium comprises an organic carbon source, such as glucose and/or glycerol, or any complex carbohydrate suitably used to grow bacterial cells.

Specifically, the batch medium further comprises ammonium salts.

According to a specific aspect, the feed medium further comprises all nutrients, supplements, and excipients as necessary to produce the fermentation product in a high-density bacterial cell culture.

Specifically, the feed medium comprises an organic carbon source, such as glucose and/or glycerol, or any chemically defined carbohydrate suitably used in a mineral medium. Specifically, the feed medium is added to the cell culture during the fed-batch phase in a growth-limiting manner.

According to a specific aspect, any one or both, the batch and feed media, further comprise trace elements.

According to a specific aspect, any one or both, the batch and feed media, have a pH ranging between 6.7 and 7.3.

According to a specific aspect, the fermentation process is performed at a pH ranging between 6.7 and 7.3, preferably wherein an aqueous ammonia or alkali solution or compound, is metered in for pH adjustment, such as to obtain the desired pH throughout the cell culture, or at least during the fed-batch phase.

Typically, the fed-batch phase begins between following about 8 to 24 hours culturing the cells in the batch phase, which may depend on various individual factors such as temperature, medium composition, medium concentration, reactor size, etc., and in particular also on the nature of the bacterial strain employed. Advantageously, synthesis of the fermentation product is switched on at about from 0 to 15 hours after beginning the fed-batch phase. However, the precise time may depend on the cell density of the culture which has already been reached at this time. By achieving a desirable cell density prior to the production phase, the volumetric yield of the desired fermentation product can be maximized.

According to a specific aspect, the host cells are grown in the batch phase to a density of at least any one of 5, 10, 20, 30, 40, or 50 g cell dry weight per liter, and the host cells are cultured in the fed-batch phase under growth-limiting conditions. Cell densities of between 10 and 80 g/l, preferably between 20 and 60 g/l, are particularly favorable.

At the start of the production phase, it is preferred that the cell density is about 10 to 60% of maximum cell density to be reached.

According to a specific aspect, the bacterial host cells are production host cells of a species selected from the group consisting of Escherichia, Pseudomonas, Bacillus, Lactococcus, Corynebacterium, Clostridium, Micrococcus, and Streptomyces.

Any suitable gram-negative bacterium may be used as the host cell for producing the fermentation product as described herein. Suitable gram-negative bacteria include Escherichia coli, Salmonella typhimurium, Pseudomonas fluorescens, Erwinia carotovora, Shigella, Klebsiella pneumoniae, Legionella pneumophila, Pseudomonas aeruginosa, and Acinetobacter baumannii. Preferably the production host cell is E. coli, in particular a recombinant E. coli engineered to produce the fermentation product at a high yield.

Examples of E. coli include those derived from Escherichia coli K12 strain, specifically, HMS174, HMS174 (DE3), XL1-Blue, C600, DH1, HB101, JM101, JM105, JM109, RV308, DH5α, XL10-Gold, TOP10, MG1655, DH10B, W3110, Origami, BW25113, as well as those derived from B-strains, specifically BL-21, BL21 (DE3), Rosetta, C41 (DE3) and the like.

Preferred bacterial host cells are recombinant cells genetically engineered to express a genetic construct for producing a fermentation product that is not produced by the wild-type (non-engineered) host cell, or to express such genetic construct at higher yields as compared to such wild-type host cells.

The fermentation product may be a heterologous product, or a product naturally-produced by a wild-type host cell, yet at a lower yield.

According to a specific aspect, the fermentation product is any one of a protein of interest (POI), metabolic pathway, RNA (such as siRNA), or a recombinant DNA molecule, such as plasmid DNA or a plasmid vector. According to a specific aspect, the fermentation product is not a plasmid DNA.

For example, the bacterial host cell can be a recombinant host cells genetically engineered to introduce a heterologous expression cassette comprising a gene of interest (GOI) that encodes a protein of interest (POI). The POI can then be produced by the bacterial host cell by expressing the GOI and isolating the POI from the cell culture. According to a specific aspect, the bacterial host cells is engineered to express a POI by incorporating one or more genes, encoding a helper protein for facilitating protein folding.

According to another example, the bacterial host cell can be genetically engineered to produce RNA or DNA molecules, including e.g., covalently closed circular (ccc) recombinant DNA molecules such as plasmids, cosmids, bacterial artificial chromosomes (BACs), bacteriophages, viral vectors and hybrids thereof.

The host cells may produce the fermentation product into the cell culture medium, or can be disrupted to release the fermentation product into the cell culture medium. The fermentation product is suitably isolated from the cell culture medium and optionally purified.

Specifically, the fermentation process includes a step of inducing the production phase. The inducing step may be through a change in the culture medium or in the cultivation technique, such as a temperature shift.

Specific embodiments employ host cells which are engineered to incorporate an inducer, such as an inducible promoter. An inducible promoter may be used that becomes activated as soon as an inductive stimulus is applied, to direct transcription of the gene under its control. Under growth conditions with an inductive stimulus, the cells usually grow more slowly than under normal conditions.

According to a specific aspect, the bacterial host cells comprise a genetic construct, in particular a heterologous genetic construct, such as an expression cassette, to produce and/or express the fermentation product, which comprises an inducible promoter, and expression of the genetic construct is induced by depletion or limitation of a cell culture component or by addition of an inducer.

Protein synthesis can be initiated by switching on a regulatable promoter system. Depending on the system which is used, this switching-on is as a rule effected by adding a substance or by altering a physical quantity.

A variety of inducible promoters can be used such as the bacterial alkaline phosphatase (phoA) promoter, tac, lac, pac, T7, T5, A1, A3, Ipp, Sp6, npr, trc, syn, σ70, pL, cspA, thrC, trp or any of a mannose, melibiose, rhamnose, or arabinose promoter.

According to a specific example, expression is induced upon phosphate depletion or limitation.

Specifically, the feed medium does not comprise phosphate. Where the phoA promoter is employed for heterologous protein expression in bacterial host cells, the cells induced for phoA promoter activity are typically starved for phosphate by culturing in a medium which gets depleted of phosphate.

According to another specific example, expression is induced using a lac system (promoter, operator and inducer), and the switching-on is carried-out by adding IPTG (isopropyl thiogalactopyranoside).

According to another specific example, plasmid-containing bacterial host cells are grown at a reduced temperature during part of the fed-batch phase, during which growth rate is restricted, followed by induction of plasmid production by a temperature up-shift and continued growth at elevated temperature in order to accumulate plasmid; the temperature shift at restricted growth rate improves yield and purity of plasmid. Such process takes advantage of the temperature sensitivity of high copy number plasmids.

According to another specific example, expression is induced by addition of a sugar (arabinose, rhamnose, melibiose, lactose or mannose)

The invention further provides for a cell culture media system for use in a bacterial fermentation process, which comprises a batch medium and a feed medium for cultivating bacterial cells. Both, the batch medium and a feed medium, are unprecipitated media solutions. Specifically, the media can be suitably used in a fed-batch fermentation process as further described herein. Specifically, the media are provided as a kit of parts or otherwise combination of a batch and feed medium, and are characterized by one or more of the features described herein with respect to the method of the invention.

Specifically:
i) the batch medium comprises an amount of calcium salts, magnesium salts, phosphate salts and at least one chelating agent, which is a citrate salt and/or citric acid at an amount of at least 5 mM, and/or EDTA at an amount of at least 1 mM; and
ii) the feed medium comprises an amount of calcium salts which is at least 1 mM, and which is at least 5-fold higher as compared to the batch medium (M/M), and
iii) the feed medium comprises an amount of magnesium salts which is at least 3 mM, and which is at least 10-fold higher as compared to the batch medium (M/M); and
iv) the feed medium comprises an amount of phosphate salts which is less than 90% of the amount comprised in the batch medium (M/M).

Specifically, the media system is characterized by any one or more of the following features:
a) the amount of calcium salts is up to 1 mM in the batch medium; and up to 5, or up to 4 mM in the feed medium;
b) the amount of magnesium salts is 1-3 mM in the batch medium; and 3-100 mM or 30-60 mM in the feed medium;
c) the amount of phosphate salts is 30-120 mM in the batch medium and in the feed medium less than 90% of the amount comprised in the batch medium (M/M);
d) the amount of the chelating agent in the batch medium is up to 100 mM citrate salt and/or citric acid; and/or up to 30 mM EDTA;
e) the batch and feed media further comprise an organic carbon source, and trace elements;
f) the batch medium further comprises a nitrogen source.

Specifically, the media are sterile solutions at a pH ranging between 6.7 and 7.3.

According to a specific example,
a) the batch medium comprises or consists of:
   (i) 0-1 mM calcium salts, preferably as CaCl₂;
   (ii) 1-3 mM magnesium salts, preferably as MgSO₄,
   (iii) 10 - 30 g/L glucose and/or glycerol;
   (iv) 50 - 125 mM ammonium, e.g. about 90 mM, preferably as one or more of (NH₄)₂SO₄, NH₄Cl, NH₄H₂PO₄, (NH₄)₂HPO₄, (NH₄)₂-H-Citrate or NH₃;
   (v) 5 - 50 mM sulphate, preferably as one or more of (NH₄)₂SO₄, MgSO₄, or K₂SO₄;
   (vi) 30 - 120 mM or 30-100 mM, phosphate, preferably as one or more of KH₂PO₄, NH₄H₂PO₄, or (NH₄)₂HPO₄ or H₃PO₄, or NaH₂PO₄, Na₂HPO₄ or K₂HPO₄;
   (vii) 5 - 100 mM citrate and/or citric acid, and/or 1 - 30 mM EDTA;
   (viii) trace elements, such as including one or more of copper, manganese, sodium, boron, zinc, and iron salts;
   (ix) and optionally an antifoam agent; and
b) the feed medium comprises or consists of:
   (i) 1-5 mM calcium salts, preferably as CaCl₂;
   (ii) 3-100 mM or 30-60 mM magnesium salts, preferably as MgSO₄;
   (iii) less than 90% of the phosphate amount contained in the batch medium (M/M), preferably as one or more of KH₂PO₄, NH₄H₂PO₄, or (NH₄)₂HPO₄ or H₃PO₄, or NaH₂PO₄, Na₂HPO₄ or K₂HPO₄;
   (iv) 500 - 800 g/L glucose and/or glycerol (e.g. the amount of glucose and glycerol in total), preferably 550-700 g/L;
   (v) trace elements, such as including one or more of copper, manganese, sodium, boron, zinc, and iron salts;
   (vi) and optionally a chelating agent, preferably up to 20 mM citrate and/or citric acid, or up to 10 mM EDTA.

The phosphate concentration in the batch medium can vary, particularly between 30 and 120 mM, or between about 30 and 100 mM. When using an expression system that is inducible upon phosphate depletion or limitation, the phosphate concentration may be lower than about 100 mM, e.g., to reduce the phosphate concentration by any one of at least 10, 20, 30, 40, 50, 60, 70, 8, or 90%.

The phosphate concentration in the feed medium can vary, and is particularly less than 90%, or less than 80%, or less than 70%, or less than 60%, or less than 50%, or less than 40%, or less than 30%, or less than 20%, or less than 10%, or less than any one of 9, 8, 7, 6, 5, 4, 3, 2, or 1%, of the concentration comprised in the batch medium (M/M). Specifically, the amount of phosphate concentration in the feed medium is reduced such that the feed medium is non-precipitating before, during and/or after autoclaving the feed medium.

The amounts, concentrations and ranges of substances contained in either medium as described herein are understood as “about” numbers +/-10%, or +/-5% of the given value.

While the batch medium comprises a certain amount of chelating agent, the feed medium may or may not comprise a chelating agent, thus, the chelating agent is only optionally present in the feed medium composition.

The invention further provides for the use of the media system described herein in a method for culturing bacterial host cells in a fed-batch fermentation process.

The invention further provides for a method for producing a fermentation product in a bacterial fermentation process, comprising:
a) a batch phase for growing the host cells; followed by
b) a fed-batch phase for producing a fermentation product from said host cells; and
c) isolating the fermentation product;
employing the media system further described herein.

The method is further characterized by the features in the context of the fermentation process and media system, as further described herein.

It was surprising that precipitation was effectively prevented when using a chelating agent in the batch medium, though there were no or only small amounts of calcium salts, and only low amounts of magnesium salts in the batch medium. By feeding calcium salts and magnesium salts during the production phase, precipitation was effectively avoided in the media and/or in the cell culture. In addition, by feeding phosphate salts in a reduced amount or with no phosphate in the feed, precipitation was effectively avoided in the media.

By the methods, fermentation process and media system as described herein consistency of fermentations was particularly improved for the phosphate-depletion induction or phosphate-limitation induction, or the stationary phase induction system (such as P-depletion system), and within a relevant pH range. In particular, phosphate precipitation was effectively avoided, thereby the performance of the fermentation and robustness has improved. Though, slight changes in pH regulation could generate more or less precipitation and therefore increase or reduce the amount of phosphate precipitates and phosphate available to the cells, the present invention provided for non-precipitating conditions throughout a pH range at least between 6.7 and 7.3, even in high cell density fermentations, yielding high titers

### DETAILED DESCRIPTION

Unless indicated or defined otherwise, all terms used herein have their usual meaning in the art, which will be clear to the skilled person. Reference is for example made to the standard handbooks, such as Sambrook et al, “Molecular Cloning: A Laboratory Manual” (2nd Ed.), Vols. 1 -3, Cold Spring Harbor Laboratory Press (1989); or Lewin, “Genes IV”, Oxford University Press, New York, (1990). Specific terms as used throughout the specification have the following meaning.

The terms “comprise”, “contain”, “have” and “include” as used herein can be used synonymously and shall be understood as an open definition, allowing further members or parts or elements. “Consisting” is considered as a closest definition without further elements of the consisting definition feature. Thus, “comprising” is broader and contains the “consisting” definition.

The term “about” as used herein refers to the same value or a value differing by +/-10% or +/-5% of the given value.

The term “cell” with respect to a “host cell” as used herein shall refer to a single cell, a single cell clone, or a cell line of a host cell.

The term “cell line” as used herein refers to an established clone of a particular cell type that has acquired the ability to proliferate over a prolonged period of time. A cell line is typically used for expressing an endogenous or recombinant nucleic acid molecule or gene, for producing fermentation products, such as to produce RNA or DNA nucleic acid molecules, products of a metabolic pathway like cell metabolites, or to produce polypeptides or proteins.

The term “host cell” as used herein shall particularly apply to any bacterial cell, which is suitably used for recombination purposes to produce a fermentation product. It is well understood that the term “host cell” does not include human beings. A “production host cell line” or “production cell line” is commonly understood to be a cell line ready-to-use for cell culture in a bioreactor to obtain the product of a fermentation process.

Specifically, recombinant host cells as described herein are artificial organisms and derivatives of native (wild-type) host cells. It is well understood that the host cells, methods and uses described herein, e.g., specifically referring to those comprising one or more genetic modifications, said heterologous expression cassettes or constructs, said transfected or transformed host cells and recombinant proteins, are non-naturally occurring, “man-made” or synthetic, and are therefore not considered as a result of “law of nature”. Genetic modifications described herein may employ tools, methods and techniques known in the art, such as described by J. Sambrook et al., Molecular Cloning: A Laboratory Manual (3rd edition), Cold Spring Harbor Laboratory, Cold Spring Harbor Laboratory Press, New York (2001).

The term “cell culture” or “culturing” or “cultivation” as used herein refers to the maintenance of cells in an in vitro, artificial environment, under conditions favoring growth, differentiation, continued viability and productivity, in an active or quiescent state of the cells, specifically in a controlled bioreactor according to methods known in the industry.

The term “cell culture medium” as referred to herein is a medium for culturing cells containing substrate and nutrients that maintain cell viability, support proliferation, growth and/or the production of a fermentation product e.g., by biotransformation of a carbon source. The cell culture medium may contain any of the following in an appropriate combination: substrate (carbon/energy source (e.g., glycerol, succinate, lactate, and sugars such as, e.g., glucose, lactose, sucrose, and fructose)), nitrogen source, precursors, and nutrients such as vitamins and minerals, salts, buffer(s), amino acids, antibiotics, serum or serum replacement, and other components such as peptide growth factors, etc.

The term “expression” or “expression cassette” is herein understood to refer to nucleic acid molecules, which contain a desired coding sequence, and control sequences in operable linkage, so that hosts transformed or transfected with these molecules incorporate the respective sequences and are capable of producing the encoded proteins or host cell metabolites. The term “gene expression”, or “expressing a polynucleotide” or “expressing a nucleic acid molecule” as used herein, is meant to encompass at least one step selected from the group consisting of DNA transcription into mRNA, mRNA processing, mRNA maturation, mRNA export, translation, protein folding and/or protein transport.

One or more expression cassettes are herein also understood as “expression system”. The expression system may be included in an expression construct, such as a vector; however, the relevant DNA may also be integrated into a host cell chromosome. Expression may refer to secreted or non-secreted expression products, including polypeptides or metabolites.

Expression cassettes are conveniently provided as expression constructs e.g., in the form of “vectors” or “plasmids”, which are typically DNA sequences that are required for the transcription of cloned recombinant nucleotide sequences, i.e., of recombinant genes and the translation of their mRNA in a suitable host organism. Expression vectors or plasmids usually comprise an origin for autonomous replication or a locus for genome integration in the host cells, selectable markers (e.g., an amino acid synthesis gene or a gene conferring resistance to antibiotics such as Zeocin, kanamycin, G418, hygromycin nourseothricin, ampicillin, chloramphenicol, tetracycline ), a number of restriction enzyme cleavage sites, a suitable promoter sequence and a transcription terminator, which components are operably linked together. The terms “plasmid” and “vector” as used herein include autonomously replicating nucleotide sequences as well as genome integrating nucleotide sequences, such as artificial chromosomes e.g., a yeast artificial chromosome (YAC).

Host cells as used herein can be obtained by introducing a vector or plasmid comprising a gene of interest into the cells. Techniques for transforming prokaryotic cells are well known in the art. These can include heat shock mediated uptake, bacterial protoplast fusion with intact cells, microinjection and electroporation.

Expression vectors may include but are not limited to cloning vectors, modified cloning vectors and specifically designed plasmids. Preferred expression vectors described herein are expression vectors suitable for expressing of a recombinant gene in a eukaryotic host cell and are selected depending on the host organism. Appropriate expression vectors typically comprise regulatory sequences suitable for expressing DNA encoding a POI in a eukaryotic host cell. Examples of regulatory sequences include promoter, operators, enhancers, ribosomal binding sites, and sequences that control transcription and translation initiation and termination. The regulatory sequences are typically operably linked to the DNA sequence to be expressed.

Examples of plasmids using gram-negative bacteria, such as Escherichia coli, as their host include pBR322, pUC18, pUC19, pUC118, pVC119, pSP64, pSP65, pTZ-18R/-18U, pTZ-19R/-19U, pGEM-3, pGEM-4, pGEM-3Z, pGEM-4Z, pGEM-5Zf(-), pET, pQE, pACYC, pBAD, and pBluescript KSTM (Stratagene). Examples of plasmids suitable for expression in Escherichia coli include pAS, pKK223 (Pharmacia), pMC1403, pMC931, and pKC30.

To allow expression of a recombinant nucleotide sequence in a host cell, a promoter sequence is typically regulating and initiating transcription of the downstream nucleotide sequence, with which it is operably linked. An expression cassette or vector typically comprises a promoter nucleotide sequence which is adjacent to the 5′ end of a coding sequence, e.g., upstream from and adjacent to the coding sequence (e.g., encoding a helper factor) or gene of interest (GOI), or if a signal or leader sequence is used, upstream from and adjacent to said signal and leader sequence, respectively, to facilitate expression and secretion of the expression product (e.g., a helper factor or the POI).

Specific expression constructs described herein comprise a promoter operably linked to a nucleotide sequence encoding a helper factor or POI under the transcriptional control of said promoter. Specifically, the promoter can be used which is not natively associated with said coding sequence.

In specific embodiments, multicloning vectors may be used, which are vectors having a multicloning site. Specifically, a desired heterologous polynucleotide can be integrated or incorporated at a multicloning site to prepare an expression vector. In the case of multicloning vectors, a promoter is typically placed upstream of the multicloning site.

The term “endogenous” as used herein is meant to include those molecules and sequences, in particular endogenous genes or proteins, which are present in the wild-type (native) host cell, prior to its modification to reduce expression of the respective endogenous genes and/or reduce the production of the endogenous proteins. In particular, an endogenous nucleic acid molecule (e.g., a gene) or protein that does occur in (and can be obtained from) a particular host cell as it is found in nature, is understood to be “host cell endogenous” or “endogenous to the host cell”. Moreover, a cell “endogenously expressing” a nucleic acid or protein expresses that nucleic acid or protein as does a host of the same particular type as it is found in nature. Moreover, a host cell “endogenously producing” or that “endogenously produces” a nucleic acid, protein, or other compound produces that nucleic acid, protein, or compound as does a host cell of the same particular type as it is found in nature.

The term “heterologous” as used herein with respect to a nucleotide sequence, construct such as an expression cassette, amino acid sequence or protein, refers to a compound which is either foreign to a given host cell, i.e. “exogenous”, such as not found in nature in said host cell; or that is naturally found in a given host cell, e.g., is “endogenous”, however, in the context of a heterologous construct or integrated in such heterologous construct, e.g., employing a heterologous nucleic acid fused or in conjunction with an endogenous nucleic acid, thereby rendering the construct heterologous. The heterologous nucleotide sequence as found endogenously may also be produced in an unnatural, e.g., greater than expected or greater than naturally found, amount in the cell. The heterologous nucleotide sequence, or a nucleic acid comprising the heterologous nucleotide sequence, possibly differs in sequence from the endogenous nucleotide sequence but encodes the same protein as found endogenously. Specifically, heterologous nucleotide sequences are those not found in the same relationship to a host cell in nature. Any recombinant or artificial nucleotide sequence is understood to be heterologous. An example of a heterologous polynucleotide is a nucleotide sequence not natively associated with a promoter, e.g., to obtain a hybrid promoter, or operably linked to a coding sequence, as described herein. As a result, a hybrid or chimeric polynucleotide may be obtained. A further example of a heterologous compound is a POI encoding polynucleotide operably linked to a transcriptional control element, e.g., a promoter, to which an endogenous, naturally-occurring POI coding sequence is not normally operably linked.

The “fermentation process” as described herein is understood as a cell culture which is fed-batch process. Specifically, a host cell, is cultured in a growth phase (in a “batch mode”) and afterwards transitioned to a production phase (“in a “fed-batch mode”) in order to produce a desired fermentation product.

A “batch phase”, or “batch mode” is to be understood as a cell culture process by which a small amount of a cell culture solution is added to a medium and cells are grown without adding an additional medium or discharging a culture solution during culture.

“Fed-batch phase” or “fed-batch mode” refers to a culture technique starting with cell growth in the batch phase, followed by a “fed” phase during which the cell culture is in continuous mode wherein the cell culture medium is continuously added (“fed”) to the bioreactor. The term “fed-batch” also includes a repeated fed-batch, and a semi-continuous fed batch fermentation process.

In a fed-batch process, either none or part of the fermentation media compounds are added to the media before the start of the fermentation and either all or the remaining part, respectively, of the compounds is fed during the fermentation process. The compounds which are selected for feeding can be fed together or separate from each other to the fermentation process.

In a repeated fed-batch process, part of the fermentation broth comprising the biomass is removed at regular time intervals.

In a semi-continuous fed batch, the complete starting medium is additionally fed during fermentation. The fermentation process is thereby replenished with a portion of fresh medium corresponding to the amount of withdrawn fermentation broth.

A growth medium used in a batch phase typically allows the accumulation of biomass, and specifically comprises a carbon source, a nitrogen source, a source for sulphur and a source for phosphate. Typically, such a medium comprises furthermore trace elements and vitamins, and may further comprise amino acids, peptone or yeast extract.

Preferred nitrogen sources include NH₄H₂PO₄, (NH₄)₂HPO₄ or NH₄Cl or (NH₄)₂-H-Citrate or NH₃ or (NH₄)₂SO_{4;}

Preferred sulphur sources include MgSO₄, or (NH₄)₂SO₄ or K₂SO_{4;}

Preferred phosphate sources include NH₄H₂PO₄, or (NH₄)₂HPO₄ or H₃PO₄, or NaH₂PO₄, KH₂PO₄, Na₂HPO₄ or K₂HPO₄;

Further typical medium components include KCl, CaCl₂, NaCl and trace elements such as: Fe, Co, Cu, Ni, Zn, Mo, Mn, I, B;

Preferably the medium is supplemented with vitamin B₁;

In the production phase, a production medium is specifically used with only a limited amount of a supplemental carbon source. For example, the feed of the supplemental carbon source added to the fermentation may comprise a carbon source with up to 50 wt% utilizable sugars, or up to 100% utilizable alcohols.

Specifically, the host cell described herein is cultured in a mineral medium with a suitable carbon source, thereby further simplifying the isolation process significantly. An example of a preferred mineral medium is one containing an utilizable carbon source (e.g., glucose, glycerol, sorbitol, methanol, ethanol, or combinations thereof), salts containing the macro elements (potassium, magnesium, calcium, ammonium, chloride, sulphate, phosphate) and trace elements (copper, iodide, manganese, molybdate, cobalt, zinc, and iron salts, and boric acid), and optionally vitamins or amino acids, e.g., to complement auxotrophies; or complex compounds, e.g. peptone, yeast extract, casein, casamino acids.

The fermentation process described herein specifically allows for the fermentation on a pilot or industrial scale in a bioreactor. A “bioreactor” can include a fermenter or fermentation unit, or any other suitable reaction vessel. The fermentation process may employ a bioreactor suitably used for industrial scale production. An industrial scale fermentation process is typically understood to encompass a fermentation process on a volume scale which is at least any one of 100 L, 500 L, 1000 L, or larger such as any one of 10, 20, 30, 40, 50, 60, 70, 80, 90, or 100 m³, or even larger up to 5000 m³.

Typical fermentation times are about 24 to 120 hours with temperatures in the range of 16° C. to 42° C., preferably 25-37° C.

The term “fermentation product” as used herein refers to the product produced by culturing a cell line in a method as disclosed herein. The fermentation product may be a compound of interest which is a nucleic acid molecule, a polypeptide or protein, including e.g., a POI, in particular heterologous proteins, or a cellular metabolite, including e.g., primary or secondary metabolites, pharmaceutical proteins or peptides, or industrial enzymes.

Primary metabolites are biomolecules that are essential to the growth, development or reproduction, and are shared by many species. Primary metabolites can be intermediates of the main metabolic pathways such as the glycolytic pathway or the TCA cycle. Examples of primary metabolites are amino acids and nucleic acids. Secondary metabolites are not essential for growth, development, or reproduction, but instead have an ecological function. Examples of secondary metabolites are antibiotics or β-lactam compounds.

The term “isolated” or “isolation” as used herein with respect to a fermentation product which may be an isolated compound of interest, shall refer to such compound that has been sufficiently separated from the environment with which it would naturally be associated, in particular a cell culture supernatant, so as to exist in “purified” or “substantially pure” form. Yet, “isolated” does not necessarily mean the exclusion of artificial or synthetic mixtures with other compounds or materials, or the presence of impurities that do not interfere with the fundamental activity, and that may be present, for example, due to incomplete purification. Isolated compounds can be further formulated to produce preparations thereof, and still for practical purposes be isolated - for example, a compound of interest can be mixed with pharmaceutically acceptable carriers or excipients when used in diagnosis or therapy.

The term “nucleic acid” used herein refers to either DNA or RNA molecules. A “polynucleotide” refers to a single or double-stranded polymer of deoxyribonucleotide or ribonucleotide bases read from the 5′ to the 3′ end. It includes expression cassettes, self-replicating plasmids, infectious polymers of DNA or RNA, and non-functional DNA or RNA.

The term “operably linked” as used herein refers to the association of nucleotide sequences on a single nucleic acid molecule, e.g., a vector, or an expression cassette, in a way such that the function of one or more nucleotide sequences is affected by at least one other nucleotide sequence present on said nucleic acid molecule. By operably linking, a nucleic acid sequence is placed into a functional relationship with another nucleic acid sequence on the same nucleic acid molecule. For example, a promoter is operably linked with a coding sequence of a recombinant gene, when it is capable of effecting the expression of that coding sequence. As a further example, a nucleic acid encoding a signal peptide is operably linked to a nucleic acid sequence encoding a POI, when it is capable of expressing a protein in the secreted form, such as a preform of a mature protein or the mature protein. Specifically, such nucleic acids operably linked to each other may be immediately linked, i.e. without further elements or nucleic acid sequences in between the nucleic acid encoding the signal peptide and the nucleic acid sequence encoding a POI.

The term “precipitate” or “precipitation” in the context of a cell culture medium is understood in the following way. Some components present at high concentration in a cell culture medium may precipitate during preparation or autoclaving or storage, especially when the pH of the medium is near neutrality. Precipitation of medium components is very undesirable because it adds an element of uncertainty. When medium components precipitate, the concentration of medium components in solution, versus in the precipitate, will be unknown. Since concentrations of various medium components can affect the quantity and quality of a fermentation product, this is an element of uncertainty that is highly undesirable in a commercial culture process, in which culture conditions are carefully controlled.

A “non-precipitating” medium is prepared as a clear solution which is devoid of particulate materials (in particular, no dispersion of solids causing turbidity), and is preferably storage-stable for at least 6 weeks to 3 months, without forming visible precipitates or a change in optical density.

As described herein, a non-precipitating batch medium (also referred to as growth medium) and/or a non-precipitating feed medium (also referred to as production medium) may be used. A non-precipitating medium system as described herein comprises at least a batch medium and a feed-medium, which are each media that are non-precipitating.

A non-precipitating medium is particularly provided as an aqueous solution comprising the respective ingredients described herein in a mixture, which is optionally autoclaved.

Specifically, a medium described herein is non-precipitating at a pH of about 7, and in particular within the pH range of 6.7 - 7.3. Specifically, a medium described herein is non-precipitating in a cell culture within the pH range of 6.7 - 7.3.

Specifically, a medium described herein is non-precipitating before and after addition of the medium to the bacterial cell culture, thereby obtaining a cell culture devoid of any non-organic turbidity or precipitates.

It is well-understood that a non-precipitating medium described herein can be provided as an aqueous medium comprising of all respective ingredients described herein, or as a kit of parts comprising at least two different media components, which may be provided to prepare a mixture of the media components before adding the mixture to the cell culture, or which may be used separately, in combination, subsequently, or in parallel, to obtain the mixture of the media components within the cell culture, at least during one of the phases of the fermentation process. One or more, e.g., all media components may be provided as a solid or respective mixture of solid materials.

Specifically, all media components of a batch medium are provided in the bacterial cell culture in the batch phase, and all media components of a feed medium are provided the bacterial cell culture in the fed-batch phase.

A “promoter” sequence is typically understood to be operably linked to a coding sequence, if the promoter controls the transcription of the coding sequence. If a promoter sequence is not natively associated with the coding sequence, its transcription is either not controlled by the promoter in native (wild-type) cells or the sequences are recombined with different contiguous sequences.

A promoter is herein described to initiate, regulate, or otherwise mediate or control the expression of a protein coding polynucleotide (DNA), such as a POI coding DNA. Promoter DNA and coding DNA may be from the same gene or from different genes, and may be from the same or different organisms.

The promoter can be an “inducible promoter” or “constitutive promoter”. The term “inducible promoter” refers to a promoter, which can be induced by the presence or absence of certain compounds or factors. Suitable promoter sequences for use with bacterial host cells, such as E. coli, include T7 promoter, T5 promoter, tryptophan (trp) promoter, lactose (lac) promoter, tryptophan/lactose (tac) promoter, lipoprotein (Ipp) promoter, and λ phage PL promoter, sugar induced promoters (arabinose, or rhamnose, or mannose, or melibiose, or lactose) in plasmids.

The term “protein of interest (POI)” as used herein refers to a polypeptide or a protein that is produced by means of recombinant technology in a host cell. More specifically, the protein may either be a polypeptide not naturally occurring in the host cell, i.e. a heterologous protein, or else may be native to the host cell, i.e. a homologous protein to the host cell, but is produced, for example, by transformation with a self-replicating vector containing the nucleic acid sequence encoding the POI, or upon integration by recombinant techniques of one or more copies of the nucleic acid sequence encoding the POI into the genome of the host cell, or by recombinant modification of one or more regulatory sequences controlling the expression of the gene encoding the POI, e.g., of the promoter sequence. In some cases, the term POI as used herein also refers to any metabolite product by the host cell as mediated by the recombinantly expressed protein.

There is no limitation with respect to the POI. The POI can be a eukaryotic or prokaryotic polypeptide, variant or derivative thereof. The protein can be a naturally secreted protein or an intracellular protein, i.e., a protein, which is not naturally secreted.

The POI can be a therapeutic or diagnostic product. Specifically, the POI is a therapeutic protein functioning in mammals. Specifically, the POI is a peptide or protein selected from the group consisting of an antigen-binding protein, a therapeutic protein, an enzyme, a peptide, a protein antibiotic, a toxin fusion protein, a carbohydrate - protein conjugate, a structural protein, a regulatory protein, a vaccine antigen, a growth factor, a hormone, a cytokine, a process enzyme, and a metabolic enzyme.

Specifically, the antigen-binding protein is selected from the group consisting of
a) antibodies or antibody fragments, such as any of chimeric antibodies, humanized antibodies, bi-specific antibodies, Fab, Fd, scFv, diabodies, triabodies, Fv tetramers, minibodies, single-domain antibodies like VH, VHH, IgNARs, or V-NAR;
b) antibody mimetics, such as Adnectins, Affibodies, Affilins, Affimers, Affitins, Alphabodies, Anticalins, Avimers, DARPins, Fynomers, Kunitz domain peptides, Monobodies, or NanoCLAMPS; or
c) fusion proteins comprising one or more immunoglobulin-fold domains, antibody domains or antibody mimetics.

A POI may be a eukaryotic protein, preferably a mammalian derived or related protein such as a human protein or a protein comprising a human protein sequence, or a bacterial protein or bacteria-derived protein. Any such mammalian, bacterial or artificial protein not naturally-occurring in the bacterial host cell is understood to be heterologous to the host cell.

The term “purified” as used herein shall refer to a preparation comprising at least 50% (mol/mol), preferably at least 60%, 70%, 80%, 90% or 95% of a compound (e.g., a POI). Purity is measured by methods appropriate for the compound (e.g., chromatographic methods, polyacrylamide gel electrophoresis, HPLC analysis, and the like). An isolated, purified compound may be obtained by purifying the cell culture supernatants to reduce impurities.

The following standard methods are preferred: cell (debris) separation and wash by Microfiltration or Tangential Flow Filter (TFF) or centrifugation, POI purification by precipitation or heat treatment, POI activation by enzymatic digest, POI purification by chromatography, such as ion exchange (IEX), hydrophobic interaction chromatography (HIC), Affinity chromatography, size exclusion (SEC) or HPLC Chromatography, POI precipitation of concentration and washing by ultrafiltration steps.

A highly purified product is essentially free from contaminating proteins, and preferably has a purity of at least 90%, more preferred at least 95%, or even at least 98%, up to 100%. The purified products may be obtained by purification of the cell culture supernatant or else from cellular debris.

As isolation and purification methods for obtaining a recombinant polypeptide or protein product, methods, such as methods utilizing difference in solubility, such as salting out and solvent precipitation, methods utilizing difference in molecular weight, such as ultrafiltration and gel electrophoresis, methods utilizing difference in electric charge, such as ion-exchange chromatography, methods utilizing specific affinity, such as affinity chromatography, methods utilizing difference in hydrophobicity, such as reverse phase high performance liquid chromatography, and methods utilizing difference in isoelectric point, such as isoelectric focusing may be used.

An isolated and purified POI can be identified by conventional methods such as Western blot, HPLC, activity assay, or ELISA.

The term “recombinant” as used herein shall mean “being prepared by or the result of genetic engineering. A “recombinant cell” or “recombinant host cell” is herein understood as a cell or host cell that has been genetically engineered or modified to comprise a nucleic acid sequence which was not native to said cell. A recombinant host may be engineered to delete and/or inactivate one or more nucleotides or nucleotide sequences, and may specifically comprise an expression vector or cloning vector containing a recombinant nucleic acid sequence, in particular employing nucleotide sequence foreign to the host. A recombinant protein is produced by expressing a respective recombinant nucleic acid in a host. The term “recombinant” with respect to a POI as used herein, includes a POI that is prepared, expressed, created or isolated by recombinant means, such as a POI isolated from a host cell transformed or transfected to express the POI. In accordance with the present invention conventional molecular biology, microbiology, and recombinant DNA techniques within the skill of the art may be employed. Such techniques are explained fully in the literature. See, e.g., Maniatis, Fritsch & Sambrook, “Molecular Cloning: A Laboratory Manual, Cold Spring Harbor, (1982). Certain recombinant host cells are “engineered” host cells which are understood as host cells which have been manipulated using genetic engineering, i.e. by human intervention. When a host cell is engineered to express a given gene or the respective protein, the host cell is manipulated such that the host cell has the capability to express such gene and protein, respectively, to a higher extent compared to the host cell under the same condition prior to manipulation, or compared to the host cells which are not engineered such that said gene or protein is expressed.

The foregoing description will be more fully understood with reference to the following examples. Such examples are, however, merely representative of methods of practicing one or more embodiments of the present invention and should not be read as limiting the scope of invention.

### EXAMPLES

### Example 1: Methods

### Medium Preparation, pH Setting and Precipitation Observation

First, medium was prepared and autoclaved. Precipitation events can be observed during media preparation, before and after autoclaving, and after adjusting the pH to 7.0. Media that were not precipitating were used for tests of cell growth in Shake flasks. The presence or absence of precipitates was determined by optical density measurement at a wavelength of 600 nm using a spectrophotometer (OD₆₀₀).

### Shake Flask Cultivations

Shake flask cultures were performed using E. coli W3110 (e.g., from DSM 5911) in order to test growth performance of media that did not precipitate during preparation. Media that allowed cells to grow in shake flask cultures were further tested at bioreactor scale.

### Bioreactor Cultivations

E. coli W3110 and LB2.0 (BL21 derived) cells were cultured in bioreactor with media that did not precipitate during preparation, autoclaving and pH adjustment, and at the same time allowed for cell growth at shake flask scale. Cell growth and product formation were monitored over time through the bioreactor cultures. Fed-batch cultures were performed in different fermentation systems and at different scales, ranging from 15 mL (ambr®15f, Sartorius), 250 mL (ambr®250, Sartorius) up to 1 L (DASGIP®, Eppendorf). Apart from precipitation, the effect of the different batch media on cell growth, maximal OD₆₀₀/DCW and titer was investigated.

### Example 2: New Media System, NPM

Several fermentations were performed to investigate the impact of the medium and its variants on growth and product formation. It surprisingly turned out that reducing calcium and magnesium salts in the batch medium and increasing the same in the feed medium, greatly improved the performance of fermentation process and reduced the tendency to produced undesired media precipitation. In addition, despite of reducing the amounts of calcium and magnesium salts in the batch medium, a chelating agent, such as citric acid, unexpectedly improved the batch medium such that its use at neutral pH up to pH 7.3, and even a higher pH, was possible without showing precipitation prior to and after autoclaving, and even during fermentation. A selection of chelating agents was used, among them sodium citrate, citric acid and EDTA. Although citrate is an additional carbon source, E. coli was not able to take-up or consume the molecule and was therefore seen to be as inert as EDTA. The cell growth proved to be not impacted from the new medium.

The new media system is termed NPM media system.

NPM media system (including a range of variants):
NPM batch medium composition:
   (i) 0-1 mM CaCl₂;
   (ii) 1-3 mM MgSO₄,
   (iii) 10 - 30 g/L glycerol;
   (iv) 50 - 125 mM (NH₄)₂SO₄ and NH₄Cl (in total);
   (v) 5 - 50 mM (NH₄)₂SO₄;
   (vi) 30 - 120 mM KH₂PO₄;
   (vii) 5 - 100 mM citrate and/or citric acid;
   (viii) trace elements, including copper chloride, manganese sulphate, sodium molybdate, boric acid, zinc sulphate, and iron sulphate;
   (ix) and an antifoam agent;
NPM feed medium composition:
   (i) 1-5 mM CaCl₂;
   (ii) 3-100 mM, preferably 30-60 mM MgSO₄;
   (iii) 550-700 g/L glycerol;
   (iv) 0-10 mM KH₂PO₄;
   (v) trace elements, including copper chloride, manganese sulphate, sodium molybdate, boric acid, zinc sulphate, and iron sulphate;
   (vi) and optionally a chelating agent: up to 20 mM citrate and/or citric acid.

### Example 3: NPM, Performance

NPM medium was compared to a precipitating medium that was proven to provide a non-limited growth and high productivity levels. The precipitating medium included a complex source, which assured the presence of all components required by the cell for growth and protein production. Therefore, it was a good reference to compare the NPM medium for similar performance but avoiding precipitation.

### Testing the NPM Combination of Batch and Feed Media Using Three Different Reporter Molecules Expressed Under Rhamnose-Induced Promoter

E. coli W3110 strains expressing three different reporter molecules under the Rhamnose promoter, were cultivated in a bioreactor in order to assess reproducibility upon performance of the NPM medium (Table 1). NPM medium was benchmarked with a complex precipitating medium (denoted “Precipitating”) in terms of growth and productivity. The reporter molecules selected were a single domain antibody (sdAb), an antibody fragment (Fab) and eGFP. The new NPM medium from the selected combination of batch and feed was also tested with complex compounds (yeast extract) in the batch.

In terms of productivity, the NPM media system, with or without addition of complex compounds, yeast extract (“NPM”, “NPM + YE”), resulted to be the best in terms of product yield, with the advantage of not having precipitation at pH 7.0.

In all cases, cell growth with the NPM medium showed no limitation and performed similarly as the complex precipitating medium.

TABLE 1

Cell growth and productivity levels of E. coli W3110 strains producing three different reporter molecules under Rhamnose-induce system in complex precipitating medium and NPM medium

| Product | Media | OD₆₀₀ [-] | DCW [g L⁻¹] | Titer pellet | Titer supernatant |
|---|---|---|---|---|---|
| Fab | Precipitating | 256 | 79 | 2.9 g L⁻¹ | 6.2 g L⁻¹ |
| Fab | NPM | 270 | 80 | 6.6 g L⁻¹ | 1.6 g L⁻¹ |
| Fab | NPM + YE | 318 | 87 | 5.5 g L⁻¹ | 1.6 g L⁻¹ |
| eGFP | Precipitating | 373 | 92 | 1.3 E7 RFU | 4.8 E5 RFU |
| eGFP | NPM | 306 | 85 | 1.2 E7 RFU | 4.2 E5 RFU |
| eGFP | NPM + YE | 373 | 89 | 1.2 E7 RFU | 4.8 E5 RFU |
| sdAb | Precipitating | 254 | 76 | 1.7 g L⁻¹ | 1.7 g L⁻¹ |
| sdAb | NPM | 235 | 71 | 2.2 g L⁻¹ | 1.6 g L⁻¹ |
| sdAb | NPM + YE | 317 | 76 | 1.7 g L⁻¹ | 1.1 g L⁻¹ |

[RFU = relative fluorescence units]

### Use of the NPM Media System With a Phosphate-Limited Promoter in Fermentation

It was further investigated whether the results provided in the Rhamnose-induced system are similar when using other promoter systems in E. coli. In this case, the effect on the phosphate depleted system was investigated in a W3110 strain producing a single domain antibody (sdAb) (Table 2). As a result, the profiles of growth and the final biomass content were comparable to the complex precipitating medium. All cultures using the NPM media system showed slightly higher single domain antibody titers and similar growth.

TABLE 2

Cell growth and productivity levels of E.coli W3110 strain producing a single domain antibody under a phosphate-limitation induction system, in complex precipitating medium and NPM medium

| Product | Media | OD₆₀₀ [-] | DCW [g L⁻¹] | Titer pellet | Titer supernatant |
|---|---|---|---|---|---|
| sdAb | Precipitating | 205 | 65 | 2.1 g L⁻¹ | 0.080 g L⁻¹ |
| sdAb | NPM | 203 | 62 | 2.6 g L⁻¹ | 0.075 g L⁻¹ |
| sdAb | NPM | 191 | 62 | 2.6 g L⁻¹ | 0.067 g L⁻¹ |

### Use of the NPM Medium in an E. Coli B Strain Producing an Antibody Fragment Under Fermentation Conditions

The performance of an E. coli B strain cultured with the NPM medium was performed. In this case, the strain LB2.0 producing an antibody fragment (Fab) under the Rhamnose-induction system was investigated in fermentation conditions. As in previous examples, the performance of the NPM medium was compared to a complex precipitating medium in terms of growth and product formation (Table 3).

TABLE 3

Cell growth and productivity levels of E.coli LB2.0 strain producing an antibody fragment using a Rhamnose-induced system, in complex precipitating medium and NPM medium

| Product | Media | OD₆₀₀ [-] | DCW [g L⁻¹] | Titer pellet [g L⁻¹] | Titer supernatant [g L⁻¹] |
|---|---|---|---|---|---|
| Fab | Precipitating | 146 | 63.7 | 1.6 | 3.7 |
| Fab | NPM | 154 | 55.6 | 2.4 | 4.2 |
| Fab | NPM | 159 | 56.6 | 1.9 | 4.4 |
| Fab | NPM | 154 | 56.6 | 1.1 | 3.8 |

### Example 4: NPM, Comparison With Media Comprising Higher Phosphate Concentration in the Feed

### Comparable: The NPM Feed Medium With High Phosphate Concentrations Is Precipitating

NPM feed medium was prepared using standard concentrations and using Glycerol as C-source. Increasing concentrations of phosphate were added to the salt solution. Once all salts were diluted, C-source (e.g. Glycerol) was added and the solutions were autoclaved at 121° C. for 30 min. Precipitation events were monitored during preparation and after autoclaving.

Concentrations of phosphate tested were 0 mM, 3 mM, 5 mM, 10 mM, 20 mM, 30 mM, 40 mM, 50 mM, 70 mM. KH₂PO₄ was used as phosphate source (Table 4).

TABLE 4

Observed precipitation during preparation of NPM feed medium with different phosphate concentrations

| | | | | | | | | | |
|---|---|---|---|---|---|---|---|---|---|
| Phosphate (mM) | 0 | 3 | 5 | 10 | 20 | 30 | 40 | 50 | 70 |
| Precipitation during preparation | No | No | No | No | No | No | No | No | No |
| Precipitation after autoclaving | No | No | No | Yes | Yes | Yes | Yes | Yes | Yes |

It is concluded that such NPM feed medium composition comprising Calcium and Magnesium salts with concentrations in the medium range, and less than 10 mM phosphate salts may be used.

In a comparable feed medium comprising Calcium and Magnesium salts with less concentrations, such as e.g., about 1 mM Calcium salts and about 3 mM Magnesium salts, higher phosphate concentrations may be used e.g., up to 108, or 105, or up to 100 mM phosphate. In any case, the feed medium may comprise less than 90% of the amount comprised in the batch medium, preferably less than 27-108 mM in the feed medium, compared to 30-120 mM in the batch medium.

### Comparables: Prior Art Media of Hardiman Et Al. (J. Biotechnol. 2007, 132:359-374) and EP1584680A1 are Precipitating

Batch and feed media from prior art were prepared as described in Hardiman et al. (J. Biotechnol. 2007, 132:359-374) and EP1584680A1 (as exemplified in Example 1). Batch medium was prepared, autoclaved and pH set to 7.0. Feed medium was prepared and autoclaved. Precipitation events were monitored during the different steps of preparation and completion (Table 5).

TABLE 5

Observed precipitation during preparation of prior art batch and feed media

| | | | | |
|---|---|---|---|---|
| | Hardiman Batch | Hardiman Feed | EP158468 0A1 Batch | EP1584680A1 Feed |
| Precipitation during preparation | No | Yes | No | No |
| Precipitation after H₃PO₄ addition | n/a | Yes | n/a | n/a |
| Precipitation after autoclaving | Yes | No | No | Yes |
| Precipitation after adjusting pH to 7.0 | Yes | n/a | No | n/a |

TABLE 6

Phosphate amounts in the media

| | Hardiman Batch | Hardiman Feed | EP158468 0A1 Batch | EP1584680A1 Feed |
|---|---|---|---|---|
| Phosphate salts (mM) | 129.9 | 479.8 | 80.1 | 79.9 |
| Amount of phosphate salts in the feed compared to the batch medium (M/M) | 369% | | 100% | |

1. A method of reducing precipitation in a bacterial fermentation process for producing a fermentation product, which fermentation process comprises the steps:
a) cultivating the host cells for bacterial growth in a batch phase using a batch medium; and
b) cultivating the host cells for producing the fermentation product in a fed-batch phase using a feed medium;
which feed and batch media are each unprecipitated media solutions, wherein:
i) the batch medium comprises an amount of calcium salts, magnesium salts, phosphate salts and at least one chelating agent, which is a citrate salt and/or citric acid at an amount of at least 5 mM, and/or EDTA at an amount of at least 1 mM; and
ii) the feed medium comprises an amount of calcium salts which is at least 1 mM, and which is at least 5-fold higher as compared to the batch medium (M/M); and
iii) the feed medium comprises an amount of magnesium salts which is at least 3 mM, and which is at least 10-fold higher as compared to the batch medium (M/M); and
iv) the feed medium comprises an amount of phosphate salts which is less than 90% of the amount comprised in the batch medium (M/M).

2. The method of claim 1, wherein
a) the amount of calcium salts is up to 1 mM in the batch medium; and up to 5, or up to 4 mM in the feed medium; and/or
b) the amount of magnesium salts is 1-3 mM in the batch medium; and 3-100 mM, or 30-60 mM in the feed medium; and/or
c) the amount of phosphate salts is 30-120 mM in the batch medium; and 0-10 mM in the feed medium.

3. The method of claim 1, wherein both, the batch and feed media further comprise an organic carbon source and trace elements.

4. The method of claim 1, wherein the batch medium further comprises ammonium salts.

5. The method of claim 1 wherein:
a) the batch medium comprises or consists of: 0-1 mM calcium salts; 1-3 mM magnesium salts; 10 - 30 g/L glucose and/or glycerol; 50 - 125 mM ammonium; 5 -50 mM sulphate; 30 - 120 mM phosphate; 5 - 100 mM citrate and/or citric acid, and/or 1 - 30 mM EDTA; trace elements; and optionally an antifoam agent; and
b) the feed medium comprises or consists of: 1-5 mM calcium salts; 3-100 mM magnesium salts; a phosphate amount which is less than 90% of the phosphate amount contained in the batch medium (M/M); 500 - 800 g/L glucose and/or glycerol; trace elements; and optionally a chelating agent.

6. The method of claim 5, wherein:
a) the batch medium is characterized by the following features:
(i) calcium salts are comprised as CaCl₂;
(ii) magnesium salts are comprised as MgSO₄,
(iii) the batch medium comprises 10 - 30 g/L glycerol;
(iv) ammonium is comprised as one or more of (NH₄)₂SO₄, NH₄Cl, NH₄H₂PO₄, (NH₄)₂HPO₄, (NH₄)₂-H-Citrate or NH₃;
(v) sulphate is comprised as one or more of (NH₄)₂SO₄, MgSO₄, orK₂SO₄;
(vi) phosphate is comprised as one or more of KH₂PO₄, NH₄H₂PO₄, (NH₄)₂HPO₄, H₃PO₄, NaH₂PO₄, Na₂HPO₄ or K₂HPO₄;
(vii) the batch medium comprises 5 - 100 mM citrate and/or citric acid;
(viii) trace elements include one or more of copper, manganese, sodium, boron, zinc, or iron salts; and
(ix) the batch medium comprises an antifoam agent; and
b) the feed medium is characterized by the following features:
(i) calcium salts are comprised as CaCl₂;
(ii) magnesium salts are comprised as MgSO₄;
(iii) the feed medium comprises 0-10 mM phosphate as one or more of KH₂PO₄, NH₄H₂PO₄, (NH₄)₂HPO₄, H₃PO₄, NaH₂PO₄, Na₂HPO₄ or K₂HPO₄;
(iv) the feed medium comprises 500 - 800 g/L glycerol;
(v) trace elements include one or more of copper, manganese, sodium, boron, zinc, or iron salts;
(vi) the feed medium comprises 0 - 20 mM citrate and/or citric acid as a chelating agent.

7. The method of claim 1, wherein the fermentation process is performed at a pH ranging between 6.7 and 7.3.

8. The method of claim 1, wherein the host cells are grown in the batch phase to a density of at least 10 g cell dry weight per liter, and the host cells are cultured in the fed-batch phase under growth-limiting conditions.

9. The method of claim 1, wherein the bacterial host cells are production host cells of a species selected from the group consisting of Escherichia, Pseudomonas, Bacillus, Lactococcus, Corynebacterium, Clostridium, Micrococcus, and Streptomyces.

10. The method of claim 1, wherein the fermentation product is any one of a protein of interest (POI), metabolic pathway, RNA, or a recombinant DNA molecule, such as plasmid DNA or a plasmid vector.

11. The method of claim 1, wherein the bacterial host cells, comprise a genetic construct to produce and/or express the fermentation product, which comprises an inducible promoter, and expression of the genetic construct is induced by limitation of a cell culture component or by addition of an inducer.

12. A media system for use in a bacterial fermentation process comprising a batch medium and a feed medium for cultivating bacterial cells, which media are each unprecipitated media solutions, wherein:
i) the batch medium comprises an amount of calcium salts, magnesium salts, phosphate salts and at least one chelating agent, which is a citrate salt and/or citric acid at an amount of at least 5 mM, and/or EDTA at an amount of at least 1 mM; and
ii) the feed medium comprises an amount of calcium salts which is at least 1 mM, and which is at least 5-fold higher as compared to the batch medium (M/M); and
iii) the feed medium comprises an amount of magnesium salts which is at least 3 mM, and which is at least 10-fold higher as compared to the batch medium (M/M); and
iv) the feed medium comprises an amount of phosphate salts which is less than 90% of the amount comprised in the batch medium (M/M).

13. The media system of claim 12, which is characterized by any one or more of the following features:
a) the amount of calcium salts is up to 1 mM in the batch medium; and up to 5, or up to 4 mM in the feed medium;
b) the amount of magnesium salts is 1-3 mM in the batch medium; and 3-100 mM, or 30-60 mM in the feed medium;
c) the amount of phosphate salts is 30-120 mM in the batch medium; and 0-10 mM in the feed medium;
d) the amount of the chelating agent in the batch medium is up to 100 mM citrate salt and/or citric acid; and/or up to 30 mM EDTA;
e) the batch and feed media further comprise an organic carbon source, and trace elements;
f) the batch medium further comprises a nitrogen source and phosphate.

14. The media system of claim 12, wherein:
a) the batch medium comprises or consists of: 0-1 mM calcium salts; 1-3 mM magnesium salts; 10 - 30 g/L glucose and/or glycerol; 50 - 125 mM ammonium; 5 - 50 mM sulphate; 30 - 120 mM phosphate; 5 - 100 mM citrate and/or citric acid, and/or 1 - 30 mM EDTA; trace elements; and optionally an antifoam agent; and
b) the feed medium comprises or consists of: 1-5 mM calcium salts; 3-100 mM magnesium salts; 0-10 mM phosphate salts; 500 - 800 g/L glucose and/or glycerol; trace elements; and optionally a chelating agent.

15. The media system of claim 14, wherein:
a) the batch medium is characterized by the following features:
(i) calcium salts are comprised as CaCl₂;
(ii) magnesium salts are comprised as MgSO₄,
(iii) the batch medium comprises 10 - 30 g/L glycerol;
(iv) ammonium is comprised as one or more of (NH₄)₂SO₄, NH₄Cl, NH₄H₂PO₄, (NH₄)₂HPO₄, (NH₄)₂-H-Citrate or NH₃;
(v) sulphate is comprised as one or more of (NH₄)₂SO₄, MgSO₄, orK₂SO₄;
(vi) phosphate is comprised as one or more of KH₂PO₄, NH₄H₂PO₄, (NH₄)₂HPO₄, H₃PO₄, NaH₂PO₄, Na₂HPO₄ or K₂HPO₄;
(vii) the batch medium comprises 5 - 100 mM citrate and/or citric acid;
(viii) trace elements include one or more of copper, manganese, sodium, boron, zinc, or iron salts;
(ix) and the batch medium comprises an antifoam agent; and
b) the feed medium is characterized by the following features:
(i) calcium salts are comprised as CaCl₂;
(ii) magnesium salts are comprised as MgSO₄;
(iii) the feed medium comprises 0-10 mM phosphate as one or more of KH₂PO₄, NH₄H₂PO₄, (NH₄)₂HPO₄, H₃PO₄, NaH₂PO₄, Na₂HPO₄ or K₂HPO₄;
(iv) the feed medium comprises 500 - 800 g/L glycerol;
(v) trace elements include one or more of copper, manganese, sodium, boron, zinc, or iron salts;
(vi) the feed medium comprises - up to 20 mM citrate and/or citric acid as a chelating agent.

16. (canceled)

17. A method for producing a fermentation product in a bacterial fermentation process, comprising:
a) a batch phase for growing the host cells; followed by
b) a fed-batch phase for producing a fermentation product from said host cells; and
c) isolating the fermentation product;
employing the media system of claim 12.

18. The method of claim 17, wherein the fermentation product is any one of a protein of interest (POI), RNA, or a recombinant DNA molecule, such as plasmid DNA or a plasmid vector.
